(19) United States
(12) Patent Application Publication
(10) Pub. No.: US 2002/0132776 A1
(43) Pub. Date: Sep. 19, 2002

(54) Inhibitors of transglutaminase

(75) Inventors: Hans-Lothar Fuchsbauer, Muhltal (DE); Ralf Pasternack, Griesheim (DE); Jens Zotzel, Darmstadt (DE)

Correspondence Address: MICHAEL BEST & FRIEDRICH, LLP, 100 E WISCONSIN AVENUE, MILWAUKEE, WI, 53202, US

(73) Assignee: N-Zyme Biotech GmbH, Darmstadt (DE)

(21) Appl. No.: 10/004,110

(22) Filed: Nov. 2, 2001

(30) Foreign Application Priority Data
Nov. 3, 2000 (DE) 10054687.0

(51) Int. Cl. A61K 38/10; A61K 38/08; A61K 38/06; A61K 38/05

(52) U.S. Cl. 514/16; 514/17; 514/18; 530/327; 530/328; 530/329; 530/330; 530/331

(57) ABSTRACT

The present invention relates to a chemical compound of the formula (I):

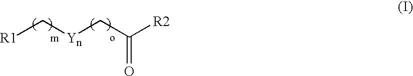

in which R¹ is:

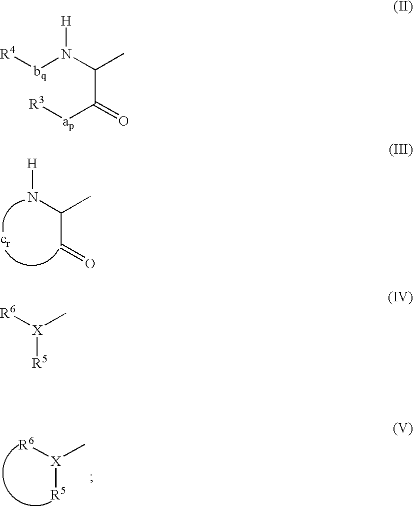

R² is H, alkyl, which may optionally be substituted by halogen or N₂, or NH₂;

m and o are 0 to 3 and n is 0 or 1;

aₚ, b_{q} and cᵣ are amino acid chains and p, q and r denote the number of amino acids, where a and/or b and/or c may likewise comprise at least one side chain represented by (CH₂)ₘYₙ(CH₂)ₒC(Z)R² where Y, Z, R², m, n, and o have the same meanings as in formula (I), and p, q and r may be identical or different and are an integer from 0 to 1000;

R³ and R⁴ are, independently of one another, H, alkyl, aryl, a heterocycle, an amino protective group or a carboxyl protective group;

R⁵ and R⁶ are, independently of one another, alkyl which may comprise at least one heteroatom selected from N, O and S, aryl or a heterocycle;

X is a methine group, a nitrogen or phosphorus atom;

Y is an oxygen atom, sulfur atom or an NH group; and

Z is an oxygen atom, sulfur atom or an NR⁷ group, where R⁷ is H, alkyl, aryl, a heterocycle, O-alkyl, O-aryl, O-heterocycle, NR₂ or NHCONR₂, where R is H, alkyl, aryl or a heterocycle, and to the use thereof as inhibitor of transglutaminases.

### CROSS-REFERENCE TO RELATED APPLICATIONS

This application claims priority under 35 U.S.C. §119 to German application no. 10054687.0 filed on Nov. 3, 2000.

### FIELD OF THE INVENTION

The present invention relates to chemical compounds as a new class of specific inhibitors of transglutaminases and to pharmaceutical compositions which comprise these compounds. The chemical compounds are suitable as inhibitors of transglutaminases and can be used to treat various diseases in which transglutaminases play a decisive part.

### BACKGROUND OF THE INVENTION

The medical relevance of transglutaminases and of the crosslinking reaction catalyzed by them in diseases have already been recognized many times and is described in detail in the relevant literature.

For example, it has been shown that the clouding of the lens of the human eye is caused by the enzymatic crosslinking of β-crystalline subunits. The activity of tissue transglutaminase is significantly raised in such cases and results in increased formation of ε-(γ-glutamyl)lysine crosslinks which make a crucial contribution to the development of cataract.

In addition, it is suggested that tissue transglutaminase is involved in diseases associated with a stimulation of the enzyme phospholipase A2 (PLA2). The transglutaminase-catalyzed modification of the phospholipase results in initiation and spread of inflammatory disorders, in particular in rheumatoid arthritis and juvenile chronic arthritis.

Recently, attention has been directed at the significance of transglutaminases in various neurodegenerative disorders, specifically in Alzheimer's disease. Important pathological characteristics thereof are the accumulation of insoluble, spiral Alzheimer fibrils within the neurons, and the extracellular amyloid deposits. Characterization of these crosslinked protein polymers and the raised transglutaminase activity are unambiguous evidence of the causal involvement of transglutaminase in dementia.

In other neurological conditions there is genetically related insertion of glutamine oligomers into proteins which are localized in the brain and thus become transglutaminase substrates. An example to be mentioned at this point is Huntington's chorea (hereditary chorea). Crosslinking occurs due to tissue transglutaminases which are likewise localized in the brain, resulting in insoluble aggregates which might, according to the medical scientific literature in its current state, cause these disorders.

A further very interesting point of attack is to deliberately inhibit transglutaminase of parasitic nematodes. The enzyme, which has only recently been sequenced and characterized in detail, plays an essential part in the development of the threadworms. There have already been promising studies in which the growth and the survival of the nematodes was reduced with comparatively nonspecific inhibitors.

The involvement of transglutaminases in apoptosis, in blood coagulation, in the development of acne, in cancer, in infections with HIV and psoriasis impressively illustrate the need for specific inhibitors for pharmaceutical use.

A simplified description of the reaction catalyzed by transglutaminases follows. The γ-carboxamide group of protein-bonded glutamine is transferred to a primary amine with liberation of ammonia. If the ε-amino function of a likewise protein-bonded lysine acts as glutamyl acceptor, the result correspondingly is an inter- or intramolecular isopeptide bond, depending on whether a second or the same peptide chain is involved as amine donor.

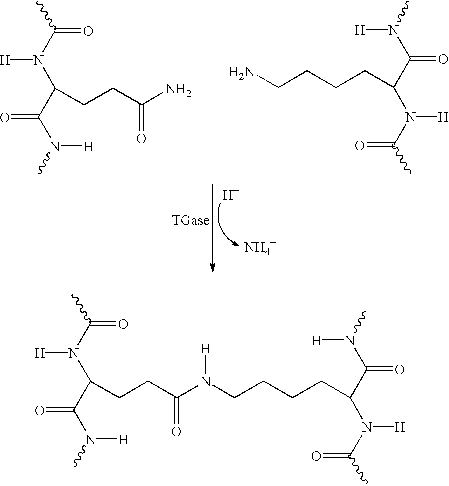

Covalent linkage of the side chains of glutamine and lysine by a transglutaminase

The ε-(γ-glutamyl)lysine isopeptide bonds in protein aggregates are not hydrolyzed in vivo by proteases. Accordingly, the crosslinking catalyzed by transglutaminases is irreversible according to the current state of knowledge.

Numerous compounds already exist for inhibiting transglutaminases but they differentiate only slightly, or not at all, between the known transglutaminases. For this reason, these inhibitors are referred to herein as nonspecific. Inhibition ordinarily takes place by reversible or irreversible blocking of the amino acids in the active site (there is primarily modification of a cysteine which is essential for the transglutaminase), after formation of the acyl-enzyme complex the binding site for peptide-bonded lysine is occupied by an amine, and the Ca²⁺ ions essential for the catalytic activity are complexed or disulfide bridges are produced by oxidative processes.

The first group of inhibitors includes iodoacetamide [Folk & Cole, J. Biol. Chem. 241, 3238-3240 (1966)], N-ethylmaleimide, para-chloromercuribenzoic acid [Folk & Cole, Biochim. Biophys. Acta, 122, 244-264 (1966)], alkyl isocyanates [Gross et al., J. Biol. Chem. 250, 7693-7699 (1975)] and other molecules with an electrophilic carbon which enter into a stable bonding with the thiol function of the cysteine. A disadvantage of these inhibitors is that they react nonspecifically with a large number of thiol groups. They accordingly have a high toxic potential because many other enzymes such as, for example, proteases may be inhibited in the same way as transglutaminases.

According to Folk [J. Biol. Chem. 244, 3707-3713 (1969)] and Chung et al. [J. Biol. Chem. 245, 6424-6435 (1970)], the binding sites for protein-bonded lysine or a primary amine is produced only after formation of the thiol ester bond between the cysteine in the active site of transglutaminase and a glutamine substrate through a change in the protein conformation. For this reason, amine inhibitors achieve their effect only when the glutamine substrate has bound to the enzyme. The demands made by transglutaminases on the lysine substrate or another amine are moreover comparatively small. Small amino compounds such as cadaverine, putrescine, spermine and spermidine (or even ammonia, added to a reaction mixture as ammonium salt) inhibit the physiological reaction by competitively occupying the resulting binding site and subsequently themselves being incorporated into the glutamine substrate. It is disadvantageous that the inhibitor must be in a distinctly higher concentration than the natural substrate for a significant inhibition to occur. In addition, amines are unsuitable, because of the mechanistic course of the catalyzed reaction, for irreversible blocking of transglutaminases or distinguishing between different transglutaminases.

The third group of inhibitors has only an effect on Ca²⁺-dependent transglutaminases. They do not block an amino acid in the active site or at another essential site in the protein but trap the bivalent cations which are necessary for the catalysis by complexation or formation of insoluble salts. These compounds include, for example, ethylenediamine-N,N,N′,N′-tetraacetic acid (EDTA), 1,2-bis-(2-aminoethoxyethane)-N,N,N′,N′-tetraacetic acid (EGTA), oxalic acid and phosphate. Such complexing agents are not suitable for pharmacological use because bivalent ions are essential in the body for a large number of complex physiological reactions.

Copper(II) salts in turn inactivate transglutaminases by oxidation of the SH groups of cysteine residues, so that cystine bridges are produced [Boothe & Folk, J. Biol. Chem. 244, 399-405 (1969)]. This inhibition relates in particular to those transglutaminases having a large number of free cysteine residues. It can be reversed by opening the cystines. Such compounds also have the disadvantage that the pharmaceutical use of heavy metals such as copper salts is associated with considerable side effects.

3,5-Substituted 4,5-dihydroisoxazoles are described in U.S. Pat. No. 4,912,120, U.S. Pat. No. 4,970,297 and U.S. Pat. No. 4,929,630 as inhibitors of transglutaminases. A particular effect ascribed to them is in the inhibition of epidermal transglutaminase and thus in the treatment of acne. Evidently, the effect of this class of substances is based on a reaction of the five-membered ring with the cysteine in the active site of transglutaminase. It is to be regarded as problematic in this connection that the dihydroisoxazole ring would, for steric reasons, be able to penetrate into the active site with considerably more difficulty than the glutamine side chain of a protein.

Oxirane compounds have been developed (U.S. Pat. No. 5,188,830) for thrombolytic therapy. These compounds are said to inhibit blood factor XIII and thus prevent the stabilization of manifest blood clots. Even if it is obvious that human factor XIII or another transglutaminase enters into stable bonding with the reactive three-membered ring, with ring opening, the described compounds appear to be unsuitable for preferential reaction with the cysteine in the active site. Other nucleophilic groups on the protein surface, such as, for example, cysteine or lysine residues, ought to enter into bonding with the oxirane ring in the same way, possibly even preferentially.

U.S. Pat. No. 4,968,713, U.S. Pat. No. 5,019,572, U.S. Pat. No. 5,021,440, U.S. Pat. No. 5,030,644, U.S. Pat. No. 5,047,416, U.S. Pat. No. 5,077,285, U.S. Pat. No. 5,084,444, U.S. Pat. No. 5,098,707, U.S. Pat. No. 5,152,988 and U.S. Pat. No. 5,177,092 describe various imidazole, pyrazole, triazole and tetrazole compounds which are likewise said to be used for the treatment of thromboses. Their mode of action is unclear.

Blocking of the active site is not obvious with other inhibitors obtained recently from biological material either. On the contrary, the inhibitors appear merely to reduce the transglutaminase activity by their binding/interaction. For example, a macrocyclic compound isolated from the culture broth of Penicillium roseopurpureum CBS 170.95 is identified as factor XIII inhibitor in U.S. Pat. No. 5,710,174 (Jan. 20, 1998).

Attempts have also been made to employ glutamine peptides with a sequence derived from the transglutaminase substrate as nontoxic inhibitors [Achyuthan et al., J. Biol. Chem. 268, 21284-21292 (1993)]. However, the inhibitory effect on factor XIII by the peptides used was comparatively small.

In a recent publication (U.S. Pat. No. 6,025,330), a more potent polypeptide from the tissue or secretions of leeches which inhibits factor XIII is now disclosed. This is a polypeptide with a molecular weight of 7000-8000 Dalton. Disadvantages compared with low molecular weight inhibitors are the elaborate purification, the high production costs and the susceptibility to proteolytic degradation, and, in particular, possible reactions of the immune system.

### SUMMARY OF THE INVENTION

It is therefore the object of the present invention to provide potent inhibitors of transglutaminases which are suitable in particular for pharmaceutical and therapeutic use. These inhibitors are intended to inhibit enzymes of the transglutaminase class in a targeted and selective manner. It was preferably intended that the inhibitors be able to distinguish specifically between two different transglutaminases in order to achieve inhibition of a particular transglutaminase type in humans and animals without abolishing the function of other endogenous transglutaminases. These chemical compounds were thus intended to be employable as potential therapeutics for the treatment of numerous disorders which are caused by transglutaminases or in which these enzymes are involved.

This object is achieved by a chemical compound of the formula (I):

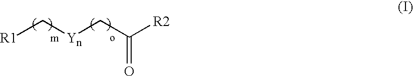

in which R¹ is:

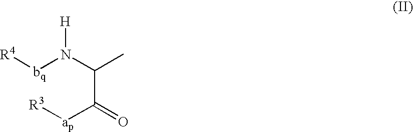

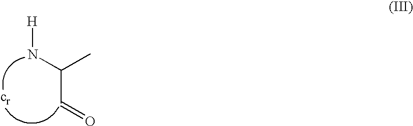

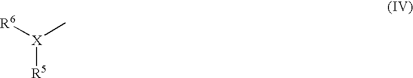

or

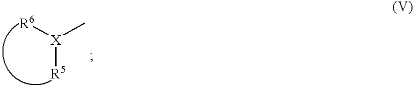

R² is H, alkyl, which may optionally be substituted by halogen or N₂, or NH₂;

m and o are 0 to 3 and n is 0 or 1;

aₚ, b_{q} and Cᵣ are amino acid chains and p, q and r denote the number of amino acids, where a and/or b and/or c may likewise comprise at least one side chain represented by (CH₂)ₘYₙ(CH₂)ₒC(Z)R² where Y, Z, R², m, n, and o have the same meanings as in formula (I), and p, q and r may be identical or different and are an integer from 0 to 1000;

R³ and R⁴ are, independently of one another, H, alkyl, aryl, a heterocycle, an amino protective group or a carboxyl protective group;

R⁵ and R⁶ are, independently of one another, alkyl which may comprise at least one heteroatom selected from N, O and S, aryl or a heterocycle;

X is a methine group, a nitrogen or phosphorus atom;

Y is an oxygen atom, sulfur atom or an NH group; and

Z is an oxygen atom, sulfur atom or an NR⁷ group, where R⁷ is H, alkyl, aryl, a heterocycle, O-alkyl, O-aryl, O-heterocycle, NR₂ or NHCONR₂, where R is H, alkyl, aryl or a heterocycle;

with the proviso that

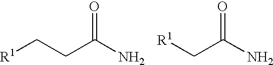

R¹ being defined as above, and

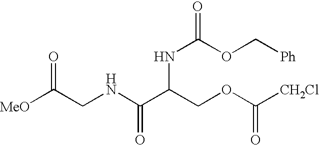

are not included.

The present invention further relates to a pharmaceutical composition comprising the chemical compound of the formula (I), and at least one other component selected from at least one pharmaceutically acceptable carrier, diluent, anticoagulant, other active ingredient and/or inhibitor.

The present invention further relates to the chemical compound of the formula (I) for use as medicament. In particular, the present invention relates to the use of the chemical compound of the formula (I) as inhibitor of transglutaminases.

### DETAILED DESCRIPTION OF THE INVENTION

The terms used herein are defined in detail below.

Halogen or Hal means fluorine, chlorine, bromine or iodine, preferably fluorine or chlorine.

Alkyl means a branched or unbranched hydrocarbon chain with 1 to 8 carbon atoms, including methyl (Me), ethyl, propyl, isopropyl, butyl, isobutyl, t-butyl, pentyl, hexyl, heptyl and octyl, where methyl, ethyl, propyl, isopropyl and t-butyl are preferred, and methyl and ethyl are particularly preferred.

Aryl means an aromatic hydrocarbon group with 6 to 10 carbon atoms, e.g. phenyl or naphthyl.

A heterocycle means a 5- or 6-membered heterocyclic monocyclic group, e.g. an oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-oxadiazol-3-yl, 1,2,4-oxadiazol-5-yl, 1,2,4-thiadiazol-3-yl, 1,2,4-thiadiazol-5-yl, 1,2,5-oxadiazol-3-yl, 1,2,5-oxadiazol-4-yl, 1,2,5-thiadiazol-3-yl, 1,2,5-thiadiazol-4-yl, 1-imidazolyl, 2-imidazolyl, 4-imidazolyl, 1-pyrrolyl, 2-pyrrolyl, 3-pyrrolyl, 2-furanyl, 3-furanyl, 2-thienyl, 3-thienyl, 2-pyridyl, 3-pyridyl, 4-pyridyl, 2-pyrimidinyl, 4-pyrimidinyl, 5-pyrimidinyl, 3-pyridazinyl, 4-pyridazinyl, 2-pyrazinyl, 1-pyrazolyl, 3-pyrazolyl and a 4-pyrazolyl group.

An amino protective group is a conventional amino protective group for amino acids. Examples include a benzyloxycarbonyl, 2-chlorobenzyloxycarbonyl, 2-brombenzyloxycarbonyl, tert-butyloxycarbonyl, pyro-glutamyl, formyl, acetyl, trifluoroacetyl, fluorenylmethoxycarbonyl, benzoyl, 2-nitrobenzoyl, 4-nitrobenzoyl, 5-N,N-dimethylaminonaphthalenesulfonyl, 4-methylbenzenesulfonyl, 2-nitrobenzenesulfonyl, 4-nitrobenzenesulfonyl group. A benzyloxycarbonyl, tert-butyloxycarbonyl or a fluorenylmethoxycarbonyl group is preferred.

A carboxyl protective group is a conventional carboxyl protective group for amino acids. Examples include a methyl, ethyl, tert-butyl, benzyl, 4-methylbenzyl, benzyloxymethyl, anisyl, thioanisyl, cresyl, thiocresyl, succinimidyl, pentafluorophenyl, diphenylmethyl, triphenylmethyl, 2,4,5-trichlorophenyl group. A methyl, ethyl, tert-butyl or benzyl group is preferred.

The chemical compound of the formula (I) is explained in detail below.

In formula (I), Y is an oxygen atom, a sulfur atom or an NH group. Y is preferably an oxygen atom.

In formula (I), m and o are 0 to 3 and n is 0 or 1. Preferably, m is 1, n is 0 or 1 and o is 0 or 1. It is particularly preferred for n to be 1 and o to be 0 when m is 1. It is preferred in this case for Y to be an oxygen atom. It is likewise preferred for n to be 0 and o to 1 when m is 1.

Z in formula (I) is an oxygen atom, a sulfur atom or an NR⁷ group, where R⁷ is defined as described above. Z is particularly preferably an oxygen atom or a sulfur atom.

R² in formula (I) is H, alkyl which can optionally be substituted by halogen or N₂, or NH₂. R² is preferably H, methyl, ethyl, t-butyl, CH₂Cl, CH₂CH₂Cl, CH₂I or CHN₂. R² is particularly preferably H, methyl, CH₂Cl or CHN₂.

In a preferred embodiment, Z is an oxygen atom and R² is H, Me, CH₂Hal or CHN₂. In another preferred embodiment, Z is a sulfur atom and R² is H, Me, CH₂Hal or CHN₂.

Preferred examples of the side chain of the chemical compound of the invention include:

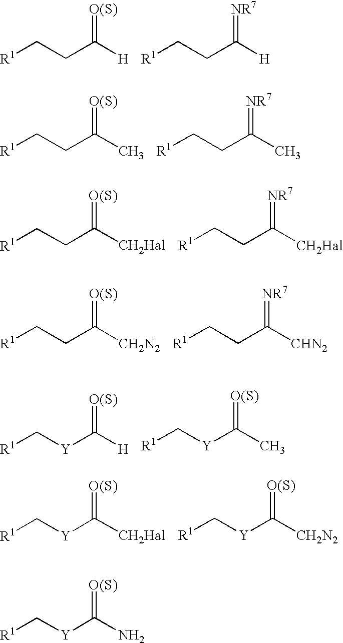

The substituent R¹ in formula (I) is

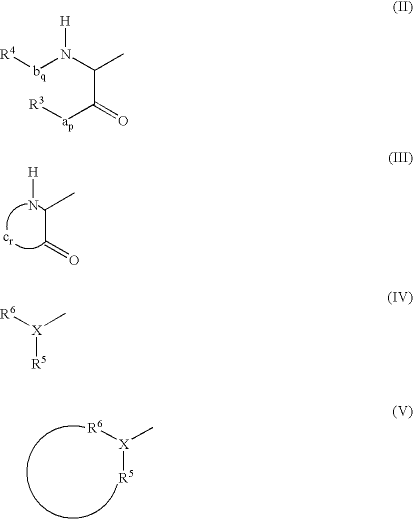

R¹ is preferably represented by formula (II).

In other words, as shown in formula (II) and (III), the chemical compound of the formula (I) may have a linear or cyclic amino acid chain aₚ, b_{q} or cᵣ.

The number of amino acids is in each case specified by p, q and r, where p and q, and r, may be identical or different and are an integer from 0 to 1000, preferably 1 to 100, more preferably 1 to 10. It is particularly preferred for p to be 1, 2, 3, 4 or 5, q to be 0, 1, 2, 3, 4 or 5 and r to be 5, 6, 7, 8, 9 or 10.

The amino acid sequences are represented by a, b and c. Conventional amino acids include alanine, valine, leucine, isoleucine, proline, tryptophan, phenylalanine, methionine, glycine, serine, tyrosine, threonine, cysteine, asparagine, glutamine, aspartic acid, glutamic acid, lysine, arginine, histidine, citrulline, homocysteine, homoserine, 4-hydroxyproline, 5-hydroxylysine, ornithine and sarcosine. Examples of possible amino acid sequences for ap are KLVFF, QKQAP, VGQPK, TPVVV, VR, KQT, RPINY, QEALP, SKIGS, EKNPL, ERQAG, QVTQT, SKVLP, MEEPA, QIV, TPVLK, QHHLG, for bq YEVHH, ICHQT, ELPEQ, IPPLT, HTTNS, KPDPS, VAAED, ALMP, FKDRV, KKTET, KETIE, GYVSS, VLSLS, DIPES, EA, KGNPE, TIGEG and for cr QHHLGTIGEG, TPVLKKGNPE, QIVEA, MEEPADIPES, SKVLPVLSLS, QVTQTGYVSS, ERQAGKETIE, EKNPLKKTET, SKIGSFKDRV, QEALPALMP, RPINYVMED, KQTKPDPS, VRHTTNS, TPVVVIPPLT, VGQPKELPEQ, QKQAPICHQT, KLVFFYEVHH.

The amino acid sequences a and/or b and/or c may likewise comprise at least one side chain represented by (CH₂)ₘYₙ(CH₂)ₒC(Z)R² where Y, Z, R², m, n, and o have the same meaning as in formula (I). Y, Z, R², m, n, and o may within a chemical compound of the formula (I) be identical or different in the respective side chains. It is preferred for all side chains (CH₂)ₘYₙ(CH₂)ₒC(Z)R² in a compound to be the same. If other side chains are present, there are normally 1 to 20, preferably 1 to 10, more preferably 1 to 3, side chains present in a and/or b and/or c.

The amino acids may be in the form of racemic mixtures or in enantiomer pure form. The L configuration is preferred. However, to prevent proteolytic degradation, D-amino acids may be incorporated in strategic positions of the polymer, e.g. at typical protease cleavage sites. This suppresses proteolytic hydrolysis of an active substance.

R³ and R⁴ in formula (i) are, independently of one another, H, alkyl, aryl, a heterocycle, an amino protective group or a carboxyl protective group. R³ and R⁴ are preferably, independently of one another, H, methyl, ethyl, tert-butyl or an amino protective group preferably selected from a benzyloxycarbonyl, tert-butyloxycarbonyl or fluorenylmethoxycarbonyl group.

R⁵ and R⁶ in the formulae (IV) and (V) are, independently of one another, alkyl which may comprise a heteroatom selected from N, O and S, preferably O, aryl or heterocycle, preferably alkyl or aryl. X in formula (IV) is a methine group, a nitrogen or a phosphorus atom, preferably a methine group.

Specific examples of the chemical compound of the formula (I) are detailed below:

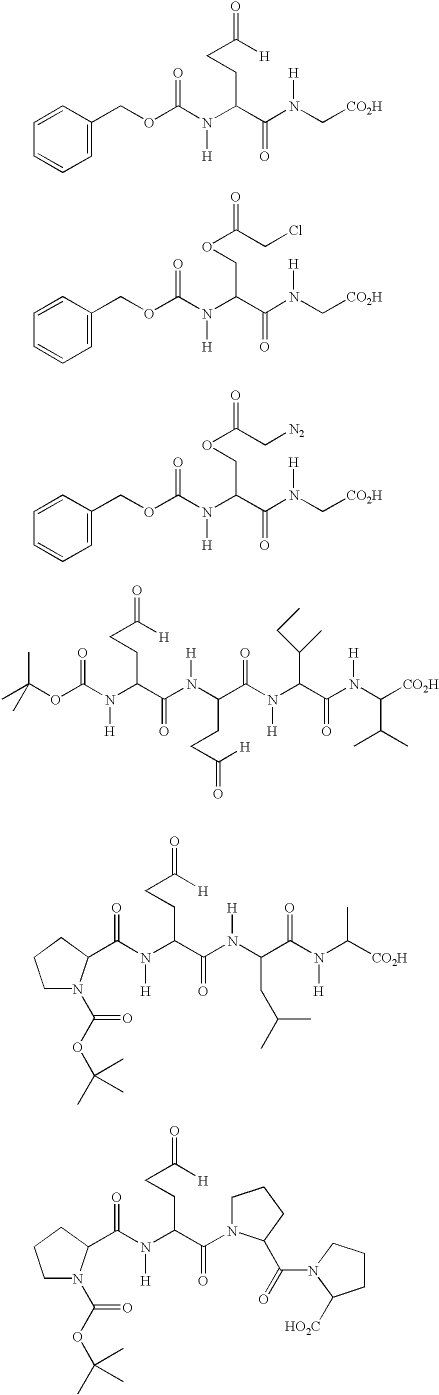

The chemical compound of the invention can be synthesized in various ways. In principle, the chemical compound in which R¹ means formula (II) or (III) is synthesized in one of two conventional ways.

In the first case there is initial molecular biological or chemical preparation of a linear or cyclic peptide with an amino chain described above. Conventional preparation processes are described, for example, in Davies, Dibner and Battey (1986) Basic methods in molecular biology, Elsevier, New York, and in Atherton and Sheppard (1989) Solid phase peptide synthesis—A practical approach, IRL Press, Oxford. The peptide is then modified by chemical or enzymatic methods (e.g. Wünsch (1974) Synthese von Peptiden in: Houben-Weyl-Muller, Methoden der Organischen Chemie XV/1 and XV/2, Thieme, Stuttgart, and Wong and Whitesides (1994) Enzymes in synthetic organic chemistry, Elsevier Science, Oxford) in such a way that the chemical compound of the invention is obtained.

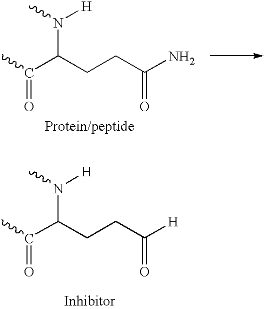

As an alternative to this, it is possible initially to synthesize, for example, from a suitable amino acid an inhibitor building block as shown by way of example in reaction sequence a) below, the inhibitor building block already having the desired functional group in the side chain, protected or unprotected.

Thereafter a linear or cyclic peptide is constructed with an aforementioned amino acid chain with incorporation of the inhibitor building block as shown by way of example in reaction sequences b) and c) below.

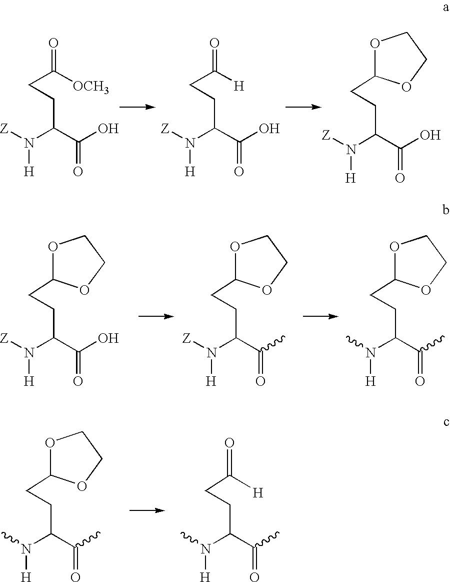

It is possible in principle for each peptide or protein to be appropriately modified in one or more amino acid side chains so that the chemical compound of the invention, in which R¹ means formulae (II) or (III), is obtained. It is likewise possible to prepare an inhibitor building block from any natural or synthetic amino acid.

Those most suitable are amino acids which can be modified by simple chemical reaction. In the first case, they are peptide-bonded, and in the second case the amino acids are free or, preferably, provided with a conventional protective group. Suitable amino acids include glutamine, glutamic acid, arginine, citrulline, ornithine, proline, serine and cysteine. Examples of modification reactions are described below.

Glutamine, glutamic acid:

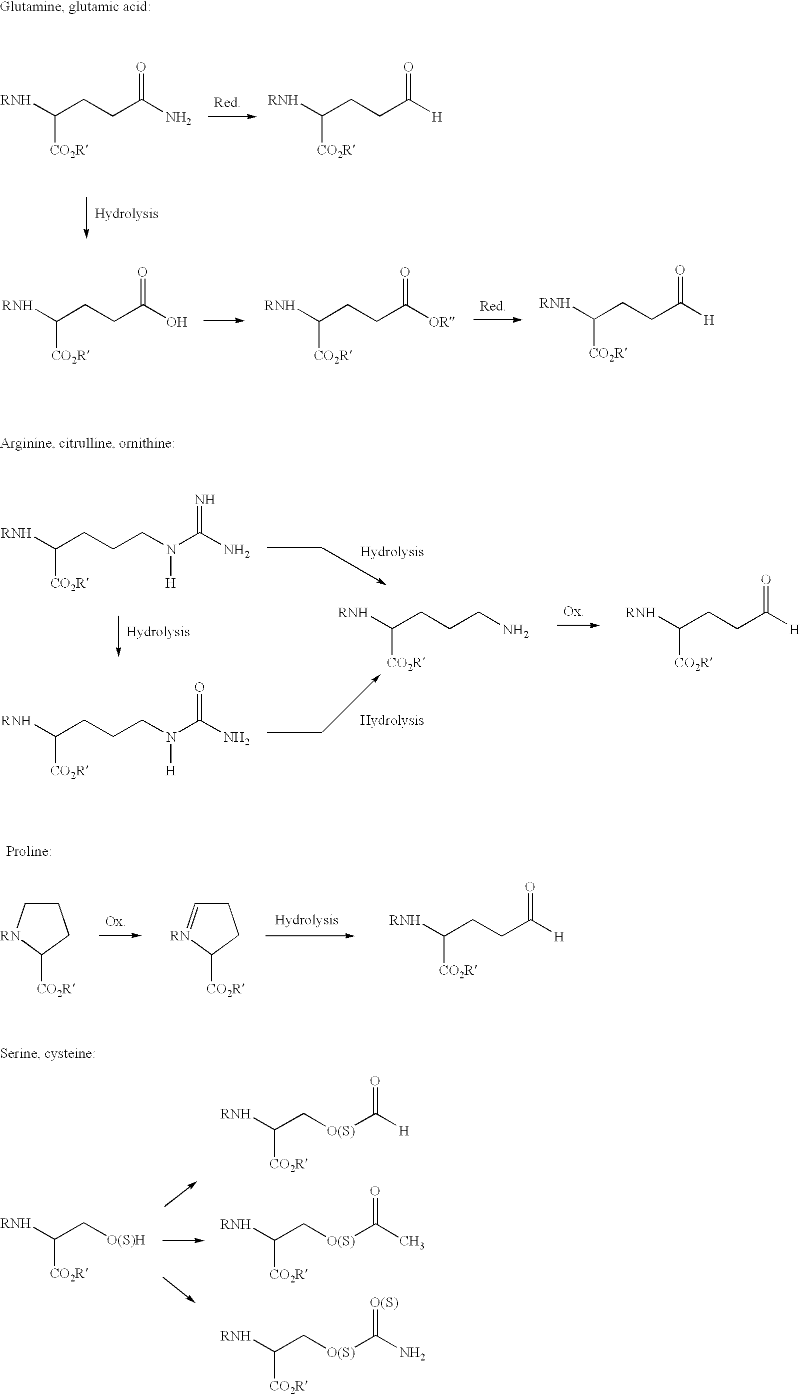

Serine, cysteine:

The chemical compound of the invention, in which R¹ means the formulae (IV) or (V), can be prepared from branched, linear or cyclic hydrocarbons with and without heteroatoms. It is merely essential that a side chain described by formula (I) and having the desired functional group is attached at the branch point X. This side chain can be introduced via a coupling reaction with base metals as described by way of example below. However, it is also possible to use other linkage reactions or other synthetic strategies which are generally known.

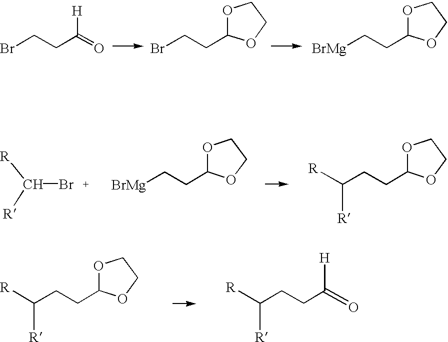

Although the chemical compound of the formula (I) of the invention can be used on its own for therapy, it is preferred to formulate the active substance in a pharmaceutical composition.

The pharmaceutical composition of the invention comprises besides the chemical compound of the formula (I) at least one other component selected from at least one pharmaceutically acceptable carrier, diluent, anticoagulant, other active ingredient and/or inhibitor.

The pharmaceutical compositions of the invention include compositions suitable for oral, rectal, nasal, topical, vaginal, intraarticular or parenteral intake, including an intramuscular, subcutaneous and intravenous intake.

The pharmaceutical composition of the invention may thus be in the form of a solid, such as tablets or filled capsules, or of a liquid, such as solutions, suspensions, emulsions, elixirs or capsules filled therewith, for oral intake; in the form of suppositories for rectal intake; or in the form of sterile injectable solutions for parenteral intake.

The pharmaceutical composition of the invention may be formulated in such a way that it permits delayed release of the compound of the formula (I). The composition may comprise conventional means with a release-slowing action for this purpose.

The pharmaceutical composition of the invention may be solid or liquid.

Solid compositions include powders, tablets, pills, capsules, including gelatin capsules, suppositories and granules.

Pharmaceutically acceptable carriers for powders and tablets include magnesium carbonate, magnesium stearate, talc, sugar, lactose, pectin, dextrin, starch, gelatin, tragacanth, methylcellulose, sodium carboxymethylcellulose and waxes with a low melting point.

Further components for powders and tablets include diluents, flavorings, solubilizers, lubricants, suspending agents, binders, preservatives, tabletting aids, disintegrants and encapsulants.

Pharmaceutically acceptable carriers which can be employed for producing suppositories comprise at least one wax with a low melting point, such as fatty acid glycerides.

Pharmaceutical compositions of the invention in liquid form include solutions, suspensions and emulsions.

Examples of the pharmaceutically acceptable carrier for parenteral use include water, aqueous propylene glycol solutions and aqueous polyethylene glycol solutions.

Further components which may be present in liquid pharmaceutical compositions include preservatives, suspending agents and dispersants, stabilizers, colorants, flavorings and thickeners.

Suitable pharmaceutically acceptable carriers for oral use include water and mixtures of water with viscous materials such as natural or synthetic gums, resins, methylcellulose, sodium carboxymethylcellulose and other known suspending agents.

Pharmaceutically acceptable carriers for topical intake are compositions with an aqueous or oily base. It is possible in this case to use as further components suitable thickeners and/or gelling agents, emulsifiers, stabilizers, dispersants, suspending agents or colorants.

The pharmaceutical composition of the invention may furthermore comprise at least one other active ingredient besides the chemical compound of the formula (I). This active ingredient is preferably a fibrinolytic, fibrinogenolytic or thrombolytic active ingredient from the group consisting of tPA, uPA, plasmin, streptokinase, eminase, hementin, hementerin, staphylokinase and bat-PA.

The pharmaceutical composition of the invention may additionally comprise besides the chemical compound of the formula (I) at least one other inhibitor. This inhibitor is preferably an inhibitor of proteases and nucleases.

The chemical compound of the invention of the formula (I) is suitable as inhibitor of transglutaminases.

It has emerged that the functional group in the side chain of formula (I) is brought in a secondary interaction via the spacer (CH₂)ₘYₙ(CH₂)ₒ into a correct position in relation to the amino acids of the catalytic triad. The spacer moreover binds to a hydrophobic pocket inside the active site and thus makes it possible for the functional group to enter into an interaction with cysteine, histidine or aspartate, preferably a covalent bonding.

It has been found that a covalent bonding with the amino acids of the catalytic triad (cysteine, histidine, aspartate) can be attained through the functional group of the chemical compound of the invention of the formula (I), because the functional group has an electrophilic center and overall a structural relationship with the γ-carboxamide function of the glutamine.

In particular, the chemical compound of the invention of the formula (I) can be used to inhibit a crosslinking of proteins and peptides, an incorporation of primary amines in proteins and peptides, a hydrolysis of the γ-carboxamide group of protein- and peptide-bonded glutamine residues, mammalian transglutaminases, human transglutaminases, blood factor XIII/blood factor XIIIa, the crosslinking of fibrin and/or α₂-plasmin inhibitor, tissue transglutaminase, liver transglutaminase, brain transglutaminase, lens transglutaminase, keratinocyte transglutaminase, epidermal transglutaminase, prostate transglutaminase, plant transglutaminase, parasitic transglutaminase and/or bacterial transglutaminase.

This makes the chemical compound of the invention of the formula (I) suitable for the treatment of numerous diseases caused by these transglutaminases or in which these enzymes are involved. For example, the chemical compound of the invention of the formula (I) can be used for the treatment of cataract, inflammatory disorders, rheumatoid arthritis, chronic arthritis, thromboses, Alzheimer's disease, Huntington's chorea, acne, cancer (induction of apoptosis), HIV infections, diseases caused by parasites, and psoriasis.

The following examples serve to illustrate the present invention.

### EXAMPLE 1

### γ-Aldehyde of carbobenzoxy-L-glutamylglycine

1.01 g (3.00 mmol) of carbobenzoxy-L-glutamylglycine are stirred under an inert gas atmosphere in 10 ml of anhydrous THF. Dropwise addition of a solution of 9 ml of 1 M LiAIH₄ in THF (9.00 mmol) and 2.67 ml (27.0 mmol) of piperidine is followed by stirring at room temperature for 20 h. The reaction mixture is poured onto ice, acidified with 10% citric acid and extracted several times with ethyl acetate. After drying over Na₂SO₄, the solvent is removed by distillation in vacuo. 0.350 g (36%) of the γ-aldehyde product is obtained.

### EXAMPLE 2

### tert-Butyloxycarbonyl-L-glutaminyl-L-alutamine Pentafluorophenyl Ester

1.00 g (2.67 mmol) of Boc-glutaminylglutamine and 0.983 g (5.34 mmol) of pentafluorophenol are dissolved with exclusion of water in 12 ml of dioxane/DMF 3:1. The reaction mixture is cooled to <5° C. and then 0.606 g (2.94 mmol) of dicyclo-hexylcarbodiimide is added in portions. The reaction mixture is then stirred for 3 h, during which it warms up to room temperature. The precipitate is removed by centrifugation, and the supernatant is evaporated to dryness in vacuo. The residue is digested with diethyl ether. 1.30 g (90%) of a colorless powder are obtained.

### EXAMPLE 3

### L-Isoleucyl-L-valine Methyl Ester

1.77 g (8.68 mmol) of para-toluenesulfonic acid monohydrate are added to a suspension of 1.00 g (4.34 mmol) of L-isoleucyl-L-valine in 50 ml of methyl acetate at room temperature. The dipeptide dissolves during this. After 4 days, the solution is extracted three times with 10 ml of saturated NaHCO₃ solution each time, washed three times with 10 ml of water each time and dried over MgSO₄. Removal of the solvent by distillation in vacuo results in 0.785 g (74%) of the colorless product.

### EXAMPLE 4

### tert-Butyloxycarbonyl-L-glutaminyl-L-glutaminyl-L-isoleucyl-L-valine Methyl Ester

0.540 g (1.00 mmol) of Boc-Gln-Gln-OPFP and 0.244 g (1.00 mmol) of Ile-Val-OMe in 10 ml of dioxane are cooled with exclusion of water to <5° C. Addition of 139 μl (1.00 mmol) of triethylamine is followed by stirring for a total of 3 h. During this, the reaction mixture warms to room temperature. The solvent is removed by distillation in vacuo, and the residue is digested with diethyl ether. 0.481 g (80%) of colorless crystals are obtained.

### EXAMPLE 5

### tert-Butyloxycarbonyl-L-glutaminyl-L-glutaminyl-L-isoleucyl-L-valine

0.601 g (1.00 mmol) of Boc-Gln-Gln-le-Val-OMe is stirred in 10 ml of 1 M NaOH at room temperature. After 2.5 h, 1 N HCl is used to neutralize. This results in a white precipitate. It is filtered off and dried over phosphorus pentoxide in vacuo. 0.230 g (39%) of the tetrapeptide is obtained.

### EXAMPLE 6

### γ-Aldehyde of tert-butyloxycarbonyl-L-glutaminyl-L-glutaminyl-L-isoleucyl-L-valine

A solution of 2 ml of 1 M LiAIH₄ in THF (2.00 mmol) and 0.6 ml (6.0 mmol) of piperidine are added dropwise to a stirred solution of 0.293 g (0.500 mmol) of Boc-Gln-Gln-Ile-Val in 5 ml of anhydrous THF under an inert gas atmosphere at room temperature. After 20 h, the reaction mixture is poured onto ice, acidified with 10% citric acid and extracted several times with ethyl acetate. Drying over Na₂SO₄ is followed by removal of the solvent by distillation in vacuo. 71.5 mg (25%) of the γ-aldehyde product are obtained.

### EXAMPLE 7

### Carbobenzoxy-L-serylglycine β-Formate

300 mg (1.01 mmol) of carbobenzoxy-L-serylglycine dissolved in 10 ml of anhydrous tetrahydrofuran are cooled with stirring to <−5° C. Then 2.06 g (10.0 mmol) of dicyclohexylcarbodiimide and 756 μl (921 mg, 20.0 mmol) of formic acid (98-100%) are added to the solution. The mixture is stirred at <−5° C. for 2 h and at room temperature for 2 d. The suspension is slowly introduced into 40 ml of ice-water, and the precipitated solid is removed by filtration through kieselguhr (Celite 521). The filtrate is extracted three times with 20 ml of ethyl acetate each time. The combined organic phases are dried over Na₂SO₄ and then the solvent is stripped off in vacuo and the resulting oil is digested with diethyl ether. 140 mg (44%) of formic ester are obtained.

¹H-NMR ([D₆]-DMSO): δ[ppm]=3.77 (m, 2H, CH₂); 4.16 (m, 1H, CH); 4.40 (m, 2H, CH₂); 5.06 (m, 2H, CH₂ (Z)); 7.39 (m, 5H, C₆H₅ (Z)); 7.73 (d, 1H, NH); 8.22 (s, 1H, HCO); 8.43 (t, 1H, NH).

ESI-MS (MeOH): m/z=347.1 (M+Na)⁺.

### EXAMPLE 8

### Carbobenzoxy-β-(O-formyl)-L-serylglycine Ethyl Ester

250 mg (0.771 mmol) of carbobenzoxy-L-serylglycine ethyl ester are dissolved with stirring in 10 ml of anhydrous tetrahydrofuran. The mixture is precooled to <−5° C. and then 816 mg (4.00 mmol) of dicyclohexylcarbodiimide and 302 μl (368 mg, 8.00 mmol) of formic acid (98-100%) are added.

The mixture is stirred at <−5° C. for 2 h and at room temperature for 2 d. The precipitated solid is then removed by filtration through kieselguhr. The filtrate is subsequently poured into 30 g of ice-water, whereupon the product precipitates immediately as a colorless solid. Precipitation is completed by storage in a refrigerator overnight. The precipitate is filtered off with suction, washed with a little ice-water and dried in vacuo over P₄O₁₀. Yield: 103 mg (37%) of the formic ester.

¹H-NMR ([D₆]-DMSO): δ[ppm]=1.18 (t, 3H, CH₃); 3.75-3.93 (m, 2H, CH₂); 4.05-4.14 (q, 2H, CH₂); 4.10-4.20 (m, 1H, CH); 4.30-4.46 (m, 2H, CH₂); 4.99-5.10 (m, 2H, CH₂ (Z)); 7.28-7.40 (m, 5H, C₆H₅ (Z)); 7.73 (d, 1H, NH); 8.21 (s, 1H, HCO); 8.55 (t, 1H, NH).

ESI-MS (MeOH): m/z=375.1 (M+Na)⁺.

### EXAMPLE 9

### Carbobenzoxy-L-serylqlycine β-acetate

100 mg (0.337 mmol) of carbobenzoxy-L-serylglycine are dissolved in 3 ml of anhydrous tetrahydrofuran and cooled to 0C. Addition of 143 μl (159 mg, 2.02 mmol) of acetyl chloride is followed by stirring at 0° C. for I h and then at room temperature for 5 days. The reaction mixture is poured into 10 g of ice-water, the oily product is taken up in 10 ml of ethyl acetate, and the aqueous phase is extracted twice with 10 ml of ethyl acetate each time. The combined organic phases are dried over Na₂SO₄ and then the solvent is removed by distillation in vacuo. The semicrystalline residue is treated with 5 ml of diethyl ether and cooled to −20° C. for several hours. The precipitate is filtered off with suction and dried in air. 55 mg (48%) of the acetylated dipeptide are obtained.

¹H-NMR ([D6]-DMSO): δ[ppm]=1.98 (s, 3H, CH₃CO); 3.66-3.87 (m, 2H, CH₂); 4.01-4.10 (m, 1H, CH); 4.22-4.29 (m, 1H, CH₂); 4.32-4.44 (m, 1H, CH₂); 5.00-5.12 (d, 2H, CH₂ (Z)); 7.30-7.40 (m, 5H, C₆H₅ (Z)); 7.68 (d, 1H, NH); 8.40 (t, 1H, NH); 12.62 (s, 1H, CO₂H).

ESI-MS (MeOH): m/z=361.1 (M+Na)⁺; 699.2 (2M+Na)⁺.

### EXAMPLE 10

### Carbobenzoxy-β-(O-acetyl)-L-serylglycine Ethyl Ester

250 mg (0.771 mmol) of carbobenzoxy-L-serylglycine ethyl ester are dissolved in 5 ml of anhydrous tetrahydrofuran and cooled to 0° C. Addition of 143 μl) (157 mg, 2.00 mmol) of acetyl chloride is followed by stirring at 0° C. for 1 h and then at room temperature for 5 days.

The reaction mixture is poured into 20 g of ice-water, and the oily product is taken up in 30 ml of ethyl acetate; the aqueous phase is extracted twice more with 20 ml of ethyl acetate each time. The combined ester extracts are dried over Na₂SO₄ and the filtrate is then evaporated to dryness in vacuo. The residue (colorless oil) is digested with 5 ml of diethyl ether and, for crystallization, stored at −20° C. for several hours. The solid is filtered off with suction and dried in air. 125 mg (44%) of the acetylated dipeptide are obtained.

¹H-NMR ([D₆]-DMSO): δ[ppm]=1.18 (t, 3H, CH₃); 1.97 (s, 3H, COCH₃); 3.74-3.95 (m, 2H, CH₂); 4.08 (q, 2H, CH₂); 4.00-4.10 (m, 1H, CH); 4.22-4.30 (m, 1H, CH₂); 4.32-4.42 (m, 1H, CH₂); 4.98-5.13 (m, 2H, CH₂ (Z)); 7.28-7.40 (m, 5H, C₆H₅ (Z)); 7.66 (d, 1H, NH), 8.50 (t, 1H, NH).

ESI-MS (MeOH): m/z=389.1 (M+Na)⁺.

### EXAMPLE 11

### Carbobenzoxy-L-serylglycine β-Chloroacetate

200 mg (0.674 mmol) of carbobenzoxy-L-serylglycine, dissolved in 5 ml of anhydrous tetrahydrofuran, are cooled to 0° C. and then 90.0 μl (126 mg; 1.12 mmol) of chloroacetyl chloride are added. The mixture is stirred at 0° C. for 2 h and at RT for 5 d. The reaction mixture is then poured into 10 g of ice-water, the oily product is taken up in 10 ml of ethyl acetate, and the remaining aqueous phase is extracted four more times with 10 ml of ethyl acetate each time. The combined ester extracts are dried over Na₂SO₄. Removal of the solvent by distillation in vacuo results in an oily residue which is digested with 5 ml of diethyl ether for crystallization. The resulting colorless solid is filtered off with suction and dried in air. Yield: 152 mg (61%) of the chloroacetic ester.

¹H-NMR ([D₆]-DMSO): δ[ppm]=3.66-3.88 (m, 2H, CH₂); 4.15-4.25 (m, 1H, CH); 4.34 (s, 2H, ClCH₂CO); 4.32-4.46 (m, 2H, CH₂); 5.00-5.12 (q, 2H, CH₂ (Z)); 7.29-7.43 (m, 5H, C₆H₅ (Z)); 7.71 (d, 1H, NH); 8.42 (t, 1H, NH); 12.68 (s, 1H, CO₂H).

ESI-MS (MeOH): m/z=373.1 (M+H)⁺; 395.1 (M+Na)⁺; 411.1 (M+K)⁺.

### EXAMPLE 12

### Carbobenzoxy-β-(O-chloroacetyl)-L-serylglycine Ethyl Ester

174 mg (0.537 mmol) of carbobenzoxy-L-serylglycine ethyl ester are dissolved in 5 ml of anhydrous tetrahydrofuran and cooled to 0° C. 160 μl (226 mg; 2.00 mmol) of chloroacetyl chloride are pipetted into this solution. The mixture is stirred at 0° C. for 3 h and at RT for 3 d.

The reaction mixture is then poured into 20 g of ice-water, whereupon the product immediately precipitates as a colorless solid. The precipitation is completed by storage in a refrigerator overnight. The precipitate is filtered off with suction, washed with a little ice-water and dried in vacuo over P₄O₁₀. Yield: 165 mg (77%) of the chloroacetic ester.

¹H-NMR ([D₆]-DMSO): δ[ppm]=1.18 (t, 3H, CH₃); 3.74-3.93 (m, 2H, CH₂); 4.08 (q, 2H, CH₂); 4.14-4.25 (m, 1H, CH); 4.34 (s, 2H, COCH₂Cl); 4.32-4.45 (m, 2H, CH₂); 5.00-5.10 (m, 2H, CH₂ (Z)); 7.28-7.40 (m, 5H, C₆H₅ (Z)); 7.70 (d, 1H, NH); 8.53 (t, 1H, NH).

ESI-MS (MeOH): m/z=423.1 (M+Na)⁺.

### EXAMPLE 13

### Chloroacetyl Derivative of the Heptapeptide pEEGSQIV

5.0 mg (6.7 μmol) pEEGSQIV are dissolved in 0.5 mL anhydrous dimethyl sulfoxide, 3 μL (4 mg; 35 μmol) chloroacetyl chloride is added and it is stirred at room temperature until the starting material is not detectable by chromatographic methods. The obtained solution is concentrated to dryness in vacuo.

### EXAMPLE 14

### Chloroacetyl Derivative of N,N-dimethylcasein

100 mg (4.40 μmol) of N,N-dimethylcasein are dissolved with stirring in 5 ml of anhydrous dimethyl sulfoxide and, at room temperature, 30.0 μl (42.0 mg, 372 μmol) of chloroacetyl chloride are added. The mixture is then stirred at room temperature for 7 d.

The resulting solution is distributed between two Centricon tubes and centrifuged at 5000× g and 25° C. for 2 h. Residues of DMSO are removed by adding 2 ml of H₂O and centrifuging at 7000× g for 2 h. The washing step is repeated twice. Lyophilization of the retentate affords 89 mg of modified casein.

### EXAMPLE 15

### Phenyl Carbonate of carbobenzoxy-L-serylcilycine Ethyl Ester

126 μl (1.00 mmol) of phenyl chloroformate are added with stirring to a cooled solution (0-5° C.) of 250 mg (0.770 mmol) of carbobenzoxy-L-serylglycine ethyl ester in 3 ml of anhydrous pyridine. The mixture is stirred at 0-5° C. for 1 h and room temperature for 2 h. The reaction mixture is poured onto 10 g of ice. The colorless precipitate is filtered off by suction and washed with a little ice-cold water. Drying over phosphorus pentoxide results in 344 mg (99%) of phenyl dipeptidyl carbonate derivative.

### EXAMPLE 16

### Carbobenzoxy-β-(O-carbamoyl)-L-serylglycine Ethyl Ester

344 mg of the phenyl dipeptidyl carbonate (Example 15) are suspended in 1 ml of anhydrous methanol while cooling in ice. Then a 7 M ammonia solution in methanol is slowly added dropwise, resulting in a clear solution after a short time. The reaction mixture is stirred at 0-5° C. for 2 h and then 10 ml of diethyl ether are added.

After cooling at −20° C. for several hours, a colorless crystalline precipitate forms and, after 12 h, is filtered off with suction, washed with 5 ml of ice-cold diethyl ether and dried in air. Yield: 100 mg (36%) of the carbamoyl compound.

¹H-NMR ([D₆]-DMSO): δ[ppm]=1.17 (t, 3H, CH₃); 3.75-3.90 (m, 2H, CH₂); 3.90-4.00 (m, 1H, CH₂); 4.02-4.12 (q, 2H, CH₂); 4.13-4.22 (m, 1H, CH); 4.27-4.38 (m, 1H, CH₂); 5.03 (s, 2H, CH₂(Z)) 6.55 (s, 2H, NH₂); 7.28-7.40 (m, 5H, C₆H₅ (Z)); 7.58 (d, 1H, NH); 8.46 (t, 1H, NH).

ESI-MS (MeCN): m/z=376.1 (M+H)⁺; 390.1 (M+Na)⁺; 406.1 (M+K)⁺.

### EXAMPLE 17

### Carbobenzoxy-L-serylglycine β-Urethane

1.00 g (3.37 mmol) of carbobenzoxy-L-serylglycine and 0.273 g (3.37 mmol) of potassium cyanate in 20 ml of anhydrous THF are cooled to <0° C. with stirring. Addition of 0.460 g (3.37 mmol) of trichloroacetic acid is followed by stirring at this temperature for 3 d. The solvent is removed by distillation in vacuo, and the residue is taken up in ethyl acetate, washed several times with water and dried over Na₂SO₄. Stripping of the solvent results in 0.743 g (65%) of the colorless urethane.

### EXAMPLE 18

### Carbobenzoxy-L-serylglycine β-Tosylate

0.5 ml (6.19 mmol) of pyridine is added dropwise to a solution of 1.00 g (3.37 mmol) of carbobenzoxy-L-serylglycine and 0.642 g (3.37 mmol) of toluenesulfonyl chloride in 20 ml of THF with exclusion of water at 0-3° C. The solution is stirred for a total of 5 h, during which it warms to room temperature. It is poured into ice-water, and the precipitate is filtered off with suction. 1.03 g (68%) of the colorless tosyl derivatives are obtained.

### EXAMPLE 19

### Carbobenzoxy-β-amino-L-alanylglycine

1.00 g (2.22 mmol) of carbobenzoxy-L-serylglycine β-tosylate is dissolved in 20 ml of ethanol. Addition of 0.5 ml (6.68 mmol) of 25% NH₃ is followed by stirring at room temperature for 2 h. Evaporation to dryness in vacuo results in 1.10 g of crude ammonium salt, which is converted without further purification into the urea derivative.

### EXAMPLE 20

### Carbobenzoxy-β-ureido-L-alanylglycine

1.10 g of crude ammonium salt of carbobenzoxy-β-amino-L-alanylglycine and 0.180 g (2.22 mmol) of potassium cyanate in 20 ml of anhydrous THF are stirred at <0° C. After addition of 0.909 g (6.66 mmol) of trichloroacetic acid, reaction is allowed to continue at this temperature for 3 d. The solvent is removed by distillation, and the residue is extracted several times with ethyl acetate. The combined organic extracts are washed with water and dried over Na₂SO₄. Removal of the solvent by distillation results in 0.338 g (45%) of the urea derivative.

### EXAMPLE 21

### Carbobenzoxy-L-asparaginylglycine tert-butyl Ester

2.32 g (6.00 mmol) of carbobenzoxy-L-asparagine para-nitrophenyl ester and 1.01 g (6.00 mmol) of glycine tert-butyl ester hydrochloride are dissolved in 12 ml of N,N-dimethylformamide and, at 0° C., 660 βl (6.00 mmol) of N-methylmorpholine are added. The mixture is stirred at 0° C. for 2 h and then at room temperature overnight. The yellow suspension is stirred into 100 ml of ice-water, and the precipitate which forms is filtered off and washed with 0.1 M HCl and water. Drying over phosphorus pentoxide is followed by recrystallization from aqueous methanol. 1.32 g (58%) of dipeptidylester are obtained.

¹H-NMR ([D₆]-DMSO): δ[ppm]=1.40 (s, 9H, (tBu)); 2.35-2.55 (m, 2H, CH₂); 3.60-3.80 (m, 2H, CH₂); 4.39 (m, 1H, CH); 5.02 (s, 2H, CH₂ (Z)); 6.90 (s, 1H, NH₂); 7.28 (s, 1H, NH₂); 7.30-7.38 (m, 5H, C₆H₅ (Z)); 7.44 (d, 1H, NH); 8.16 (t, 1H, NH).

### EXAMPLE 22

### Carbobenzoxy-β-(N-carbamoyl)-L-aminoalanylglycine tert-butyl Ester

312 mg (0.823 mmol) of carbobenzoxy-L-asparaginylglycine tert-butyl ester and 387 mg (0.900 mmol) of [bis-(trifluoroacetoxy)iodo]benzene are dissolved with stirring in 20 ml of acetonitrile/H₂O (1:1). Addition of 245 μl of pyridine is followed by stirring overnight. Then acetonitrile is completely stripped off in vacuo, and the remaining aqueous solution is extracted three times with 10 ml of diethyl ether each time. Acidification of the aqueous phase with 10% acetic acid to pH 2.8 is followed by addition of 214 mg (3.30 mmol) of sodium cyanate at room temperature and stirring for 2 d. Extraction with ethyl acetate, drying over Na₂SO₄ and removal of the solvent by distillation afford 102 mg (31%) of a colorless crystalline solid.

¹H-NMR ([D₆]-DMSO): δ[ppm]=1.41 (s, 9H, (tBu)); 3.09 (m, 1H, CH₂); 3.40 (m, 1H, CH₂); 3.72 (m, 2H, CH₂); 4.03 (m, 1H, CH); 5.04 (s, 2H, CH₂ (Z)); 5.67 (s, 2H, NH₂); 6.02 (t, 1H, NH); 7.30-7.40 (m, 5H, C₆H₅ (Z)); 7.46 (d, 1H, NH); 8.27 (t, 1H, NH).

ESI-MS (MeOH): m/z=395.2 (M+H)⁺; 417.2 (M+Na)⁺; 433.1 (M+K)⁺; 811.4 (2M+Na)⁺.

### EXAMPLE 23

### Carbobenzoxy-β-(N-carbamoyl)-L-aminoalanylglycine

102 mg (0.259 mmol) of carbobenzoxy-β-(N-carbamoyl)-L-aminoalanylglycine tert-butyl ester are dissolved in 5 ml of trifluoroacetic acid at room temperature and then stirred at this temperature for 30 min. The solution is concentrated to about 1 ml in vacuo at 40° C., and digested with 5 ml of diethyl ether and cooled at −20° C. overnight.

The crystals are filtered off with suction and washed with a little ice-cooled diethyl ether. Drying in air affords a yield of 64 mg (73%) of colorless solid.

¹H-NMR ([D₆]-DMSO): δ[ppm]=3.05-3.15 (m, 1H, CH₂); 3.30-3.45 (m, 1H, CH₂); 3.69-3.78 (m, 2H, CH₂); 4.04 (m, 1H, CH); 5.03 (s, 2H, CH₂ (Z)); 5.64 (s, 2H, NH₂); 6.00 (t, 1H, NH); 7.28-7.40 (m, 5H, C₆H₅ (Z)); 7.45 (d, 1H, NH); 8.22 (t, 1H, NH); 12.58 (s, 1H, CO₂H).

ESI-MS (MeOH): m/z=339.2 (M+H)⁺; 361.1 (M+Na)⁺

### EXAMPLE 24

### Carbobenzoxy-L-(tert-butyl)glutamylglycine Benzyl Ester

5.00 g (11.5 mmol) of succinimidyl carbobenzoxy-L-(tert-butyl)glutamate are dissolved with stirring in 20 ml of tetrahydrofuran. A solution of 2.32 g (11.5 mmol) of glycine benzyl ester hydrochloride in 25 ml of 1 M NaHCO₃ are added and the mixture is stirred at room temperature overnight.

The solvent is then removed by distillation. The oily product is taken up in 50 ml of ethyl acetate, and the aqueous phase is extracted twice with 25 ml of ethyl acetate each time. The combined organic phases are dried over Na₂SO₄. Stripping off the solvent results in 5.54 g (99%) of a colorless oil. This crude product is digested with 4 ml of diethyl ether and, for crystallization, cooled at −20° C. for some hours. The colorless crystals are filtered off with suction, washed with a little ice-cold ether and dried in air. Yield: 4.01 g (72%).

¹H-NMR ([D₆]-DMSO): δ[ppm]=1.38 (s, 9H, tBu); 1.65-1.82 (m, 1H, CH₂); 1.82-1.98 (m, 1H, CH₂); 2.27 (t, 2H, CH₂); 3.80-4.02 (m, 2H, CH₂); 4.00-4.10 (m, 1H, CH); 4.97-5.09 (m, 2H, CH₂ (Z)); 5.12 (s, 2H, CH₂ (Bzl)); 7.28-7.40 (m, 10H, C₆H₅ (Z,Bzl)); 7.49 (d, 1H, NH); 8.39 (t, 1H, NH).

### EXAMPLE 25

### Carbobenzoxy-L-glutamylglycine Benzyl Ester

2.63 g (5.43 mmol) of carbobenzoxy-L-(tert-butyl)glutamylglycine benzyl ester are dissolved with stirring in 5 ml of anhydrous dichloromethane. Then 20 ml of trifluoroacetic acid are added dropwise over a period of 5 min, and the mixture is stirred for a further 25 min. The solution is subsequently concentrated to 2-3 ml in vacuo at 40° C. The residue is mixed with 15 ml of diethyl ether, and crystallization occurs on cooling to room temperature. The crystallization is completed by cooling the suspension at −20° C. for some hours.

The crystals are filtered off with suction and washed with several portions of ice-cold diethyl ether and then dried in air. 2.18 g (94%) of colorless crystalline solid are obtained.

¹H-NMR ([D₆]-DMSO): δ[ppm]=1.70-1.85 (m, 1H, CH₂); 1.85-2.00 (m, 1H, CH₂); 2.30 (t, 2H, CH₂); 3.92 (m, 2H, CH₂); 4.08 (m, 1H, CH); 4.97-5.09 (m, 2H, CH₂ (Z)); 5.14 (m, 2H, CH₂ (Bzl)); 7.30-7.42 (m, 10H, C₆H₅ (Z,Bzl)); 7.52 (d, 1H, NH); 8.41 (t, 1H, NH).

### EXAMPLE 26

### Carbobenzoxy-L-6-diazo-5-oxonorleucylglycine Benzyl Ester

1.01 g (2.33 mmol) of carbobenzoxy-L-glutamylglycine benzyl ester are dissolved in 10 ml of dry tetrahydrofuran, and the solution is cooled to −20° C. Addition of 312 μl (328 mg, 2.40 mmol) of isobutyl chloroformate and 264 μl (243 mg, 2.40 mmol) of N-methylmorpholine is followed by stirring at <−20° C. for 15 min. The reaction mixture is then slowly added dropwise to an etheral diazomethane solution (50 ml) so that the temperature does not exceed 0° C. 30 min at 0° C. are followed by stirring at room temperature overnight. After the solvent has been stripped off in vacuo, the crude diazomethyl ketone is obtained as a brown oil which is chromatographed on silica gel 60 (eluent: n-hexane/ethyl acetate 2:1). Yield: 516 mg (47%) of colorless crystals.

### EXAMPLE 27

### Carbobenzoxy-L-6-chloro-5-oxonorleucylglycine Benzyl Ester

1.20 g (2.80 mmol) of carbobenzoxy-L-glutamylglycine benzyl ester are dissolved in 10 ml of anhydrous tetrahydrofuran and cooled to <−20° C. Addition of 375 μl (292 mg, 2.88 mmol) of isobutyl chloroformate and 317 μl (292 mg, 2.88 mmol) of N-methyl-morpholine is followed by stirring at <−20° C. for 15 min. The reaction mixture is then slowly (over 10 min) added dropwise to a diazomethane solution in ether (60 ml) and then stirred at 0° C. for 30 min and at room temperature for 16 h. A 1 M HCl solution (in ether) is added dropwise to this solution until an pH of 3 is reached. The solution is finally treated quickly with saturated NaHCO₃ solution, and the etherial phase is dried over magnesium sulfate. Removal of the solvent by distillation results in the chloromethyl ketone as colorless oil (890 mg, 69%).

### EXAMPLE 28

### γ-Aldehyde of carbobenzoxy-L-glutamylglycine

A stirred solution of 1.13 mL (2.26 μmol) oxalyl chloride in 10 mL anhydrous dichloromethane is cooled to −78° C. and 0.35 mL dimethyl sulfoxide is slowly added. After 5 minutes it is dropwise added in this sequence a solution of 765 mg (2.26 μmol) L-2-carbobenzoxyamino-5-hydroxyvalerylglycine methyl ester in 2 mL dichloromethane and a solution of 1.5 mL triethylamine in dichoromethane. After a further 5 minutes at −78° C. the reaction mixture is warmed to 0° C. and partitioned between dichloromethane and water. The organic phase is dried over Na₂SO₄ and the solvent is distilled. As the residue, the aldehyde (350 mg, 46%) is obtained as a colorless oil.

### EXAMPLE 29

### Inhibition of Bacterial Transglutaminase and Tissue Transglutaminase

The inhibitors are dissolved in dimethyl sulfoxide (DMSO) at a concentration of 100 mM. Solutions having a concentration of 10 mM, 1 mM and 0.1 mM are prepared by dilution with transglutaminase buffer (50 mM Tris-HCl, pH 7.0, 5 mM CaCl₂, 2 mM DTT).

Afterwards, inhibition takes place by incubation (20 minutes at 37° C.) of 25 μL of a transglutaminase solution (bacterial transglutaminase or guinea pig liver transglutaminase, respectively) with 25 μL of the respective inhibitor solution. Immediately after the incubation has finished, 100 μL of substrate solution (0.1 mM hydroxylamine, 30 mM CBZ-Gln-Gly-OH, 2 mM DTT, 5 mM CaCl₂ in 50 mM Tris-HCl, pH 7.0) are added and it is incubated for 10 minutes at 37° C. The reaction is stopped with 100 μL of a solution of 4% (w/v) HCl, 1.7% (w/v) FeCl₃ and 4% (w/v) trichloroacetic acid [see Grossowicz et al., J. Biol. Chem. 187, 111-125, 1950]. After centrifugation (10 minutes, 10,000× g) the extinction of the supernatant solution at 492 nm in the microtiter plate reader and, thus, the remaining activity of the transglutaminase is determined.

| | Inhibition of tissue | | |
|---|---|---|---|
| | transglutaminase at an | | |
| | inhibitor concentration of | | |
| Tested substance | 5 mM | 0.5 mM | 0.05 mM |
| Carbobenzoxy-L-serylglycine β- | 56% | 0% | 0% |
| formate | | | |
| Carbobenzoxy-L-serylglycine β- | 0% | 0% | 0% |
| acetate | | | |
| Carbobenzoxy-L-serylglycine β- | 91% | 43% | 1% |
| chloroacetate | | | |
| Carbobenzoxy-β-(O-formyl)-L- | 0% | 0% | 0% |
| serylglycine ethyl ester | | | |
| Carbobenzoxy-β-(O-acetyl)-L- | 0% | 0% | 0% |
| serylglycine ethyl ester | | | |
| Carbobenzoxy-β-(O-chloroacetyl)-L- | 76% | 7% | 2% |
| serylglycine ethyl ester | | | |
| Carbobenzoxy-β-(O-carbamoyl)-L- | 0% | 0% | 0% |
| serylglycine ethyl ester | | | |
| Carbobenzoxy-β-(N-carbamoyl)-L- | 0% | 0% | 0% |
| aminoalanylglycine tertbutyl ester | | | |
| Carbobenzoxy-β-ureido-L- | 35% | 0% | 0% |
| alanylglycine | | | |
| Carbobenzoxy-L-serylglycine β- | 72% | 7% | 5% |
| formate | | | |
| Carbobenzoxy-L-serylglycine β- | 34% | 2% | 3% |
| acetate | | | |
| Carbobenzoxy-L-serylglycine β- | 99% | 98% | 29% |
| chloroacetate | | | |
| Carbobenzoxy-β-(O-formyl)-L- | 30% | 17% | 7% |
| serylglycine ethyl ester | | | |
| Carbobenzoxy-β-(O-acetyl)-L- | 31% | 15% | 6% |
| serylglycine ethyl ester | | | |
| Carbobenzoxy-β-(O-chloroacetyl)-L- | 100% | 88% | 16% |
| serylglycine ethyl ester | | | |
| Carbobenzoxy-β-(O-carbamoyl)-L- | 22% | 12% | 3% |
| serylglycine ethyl ester | | | |
| Carbobenzoxy-β-(N-carbamoyl)-L- | 0% | 14% | 7% |
| aminoalanylglycine tertbutyl ester | | | |
| Carbobenzoxy-β-ureido-L- | 49% | 0% | 0% |
| alanylglycine | | | |

### Example 31

### Inhibition of Guinea Pig Liver Transglutaminase and Factor XIIIa by the γ-Aldehyde of carbobenzoxy-L-glutamylglycine

Factor XIIIa:

The inhibitor tests were carried out in microtiter plates (0.2 ml). This entailed examination of the ability of the plasma transglutaminase to catalyze the coagulation of fibrin with and without inhibitor [modified method of Tymiak et al., J. Antibiot., 46, 204-206 (1993)].

40 μl of bovine fibrinogen (3 mg/ml, Sigma) in 25 mm Tris-HCl with 100 mm NaCl and 2.5 mm Ca²⁺, pH 7.5, were incubated with 2 U/ml bovine thrombin (Sigma) at 25° C. for 10 min.

In a second mixture in parallel therewith, likewise 2 U/ml bovine thrombin (Sigma) were added to 20 μg/ml FXIII in 25 mm Tris-HCl with 100 mm NaCl and 2.5 mm Ca²⁺, pH 7.5, and incubated at 25° C. for 10 min. The γ-aldehyde inhibitor concentration was then adjusted so that the measurements covered a concentration range from 0 to 10 μm. The reaction mixture was incubated at 25° C. for 15 min.

The two mixtures were combined and, after an incubation at 30° C. for 15 min, 150 μl of urea (8 M) were added. The absorption of the clots was measured at 405 nm.

This showed that factor XIIIa cannot be inhibited by the γ-aldehyde of carbobenzoxy-L-glutamylglycine.

Guinea Pig Liver Transglutaminase:

The inhibitor tests were carried out in analogy to the factor XIII test in microtiter plates. In this case, the incorporation of hydroxylamine into carbobenzoxy-L-glutaminylglycine (CBZ-Gln-Gly) was investigated [modified method of Grossowicz et al., J. Biol. Chem., 187, 111 -125 (1950)].

25 μl (0-10 μM) of inhibitor were added to 25 μl (5.0 U/ml) of guinea pig liver transglutaminase (Sigma) and incubated at 25° C. for 15 min. Then 100 μl of 0.2 M tris-acetate buffer, pH 6.0, with 30 mm CBZ-Gln-Gly, 0.1 M hydroxylamine and 10 mM glutathione were pipetted in. After 10 min at 37° C., the absorption at 492 nm was measured using a microtiter plate reader.

Guinea pig liver transglutaminase is inhibited by as little as 1 μM γ-aldehyde of carbobenzoxy-L-glutamylglycine.

### EXAMPLE 32

### Inhibition of Factor XIIIa by the γ-Aldehyde of tert-butyloxycarbonvl-L-glutaminyl-L-glutaminyl-L-isoleucyl-L-valine

The test was carried out as described in Example 31 (factor XIIIa). The tetrapeptide was able to suppress fibrin crosslinking. The γ-aldehyde of tert-butyloxycarbonyl-L-glutaminyl-L-glutaminyl-L-isoleucyl-L-valine is thus an effective factor XIIIa inhibitor.

We claim:

1. A chemical compound of the formula (I): 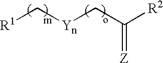
in which R¹ is: 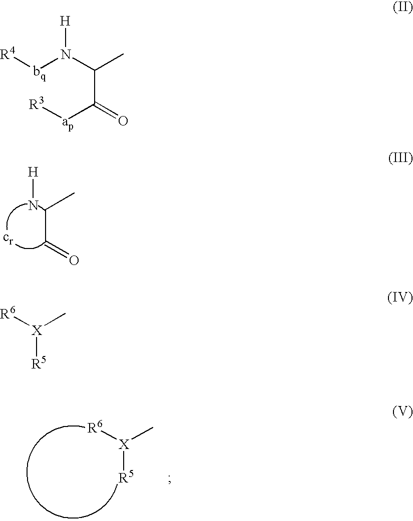
R² is H, alkyl, which may optionally be substituted by halogen or N₂, or NH₂;
m and o are 0 to 3 and n is 0 or 1;
aₚ, b_{q} and cᵣ are amino acid chains and p, q and r denote the number of amino acids, where a and/or b and/or c may likewise comprise at least one side chain represented by (CH₂)ₘYₙ(CH₂)ₒC(Z)R² where Y, Z, R², m, n, and o have the same meanings as in formula (I), and p, q and r may be identical or different and are an integer from 0 to 1000;
R³ and R⁴ are, independently of one another, H, alkyl, aryl, a heterocycle, an amino protective group or a carboxyl protective group;
R⁵ and R⁶ are, independently of one another, alkyl which may comprise at least one heteroatom selected from N, O and S, aryl or a heterocycle;
X is a methine group, a nitrogen or phosphorus atom;
Y is an oxygen atom, sulfur atom or an NH group; and
Z is an oxygen atom, sulfur atom or an NR⁷ group, where R⁷ is H, alkyl, aryl, a heterocycle, O-alkyl, O-aryl, O-heterocycle, NR₂ or NHCONR₂, where R is H, alkyl, aryl or a heterocycle;
with the proviso that 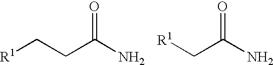
R¹ being defined as above, and 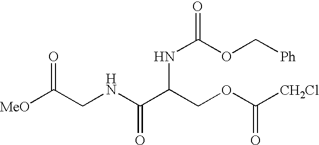
are not included.

2. The chemical compound as claimed in claim 1, wherein m is 1, n is 0 or 1 and o is 0 or 1.

3. The chemical compound as claimed in claim 1, wherein R² is H, Me, CH₂Hal or CHN₂ when Z is an oxygen atom.

4. The chemical compound as claimed in claim 1, wherein R² is H, Me, CH₂Hal or CHN₂ when Z is a sulfur atom.

5. A pharmaceutical composition comprising the chemical compound as claimed in claim 1 and a component, the component being at least one of a pharmaceutically acceptable carrier, a diluent, an anticoagulant, an active ingredient and an inhibitor.

6. The pharmaceutical composition as claimed in claim 5, wherein the active ingredient is a fibrinolytic, fibrinogenolytic or thrombolytic active ingredient from the group consisting of tPA, uPA, plasmin, streptokinase, eminase, hementin, hementerin, staphylokinase and bat-PA.

7. A method of medicating a mammal, the method comprising:
administering an effective dosage of the chemical compound as claimed in claim 1; and
medicating the mammal.

8. A method of inhibiting transglutaminase in a mammal, the method comprising:
administering an effective amount of the chemical compound as claimed in claim 1 to a mammal; and
inhibiting transglutaminase in the mammal.

9. The method of claim 8, wherein at least one of the following is inhibited:
crosslinking of proteins and peptides,
incorporation of primary amines in proteins and peptides,
hydrolysis of the γ-carboxamide group of protein- and peptide-bonded glutamine residues,
mammalian transglutaminases,
human transglutaminases,
blood factor XIII/blood factor XIIIa,
crosslinking of fibrin and/or a₂-plasmin inhibitor,
tissue transglutaminase,
liver transglutaminase,
brain transglutaminase,
lens transglutaminase,
keratinocyte transglutaminase,
epidermal transglutaminase,
prostate transglutaminase,
plant transglutaminase,
parasitic transglutaminase and
bacterial transglutaminase.

10. The method of claim 8, wherein at least one of the following is treated: a cataract, inflammatory disorders, rheumatoid arthritis, chronic arthritis, thromboses, Alzheimer's disease, Huntington's chorea, acne, cancer (induction of apoptosis), HIV infections and psoriasis.
